(19) United States
(12) Patent Application Publication
(10) Pub. No.: US 2024/0274236 A1
(43) Pub. Date: Aug. 15, 2024

(54) MOLECULAR SEQUENCE DESIGN TOOLS

(71) Applicant: Benchling, Inc., San Francisco, CA (US)

(72) Inventors: Pathikrit Bhattacharyya, Oakland, CA (US); Lily Helfrich, Oakland, CA (US)

(21) Appl. No.: 18/440,865

(22) Filed: Feb. 13, 2024

Related U.S. Application Data
(60) Provisional application No. 63/445,188, filed on Feb. 13, 2023.

(51) Int. Cl. G16B 30/20; G16B 30/10; G16B 45/00

(52) U.S. Cl. CPC G16B 30/20; G16B 30/10; G16B 45/00

(57) ABSTRACT

Methods, systems, and apparatus, including computer programs encoded on a computer storage medium, for automatically generating molecular constructs. One of the methods includes generating a user interface presentation for automatically generating different combinations of molecular sequences, wherein the user interface presentation presents a sequence of bins for generating a molecular construct. Each bin is associated with data representing a respective plurality of fragments for generating the molecular construct. A plurality of different construct combinations are generated representing different variations of the construct, including selecting, for each different construct combination, one fragment from each bin in the sequence of bins.

### CROSS-REFERENCE TO RELATED APPLICATION

This application claims priority to U.S. Provisional Application No. 63/445,188, filed on Feb. 13, 2023. The disclosure of the prior application is considered part of and is incorporated by reference in the disclosure of this application.

### BACKGROUND

This specification relates to software tools for visualizing and designing molecular sequences.

Modern research and development operations in the life sciences often engage in high-throughput sequence design of molecular material, e.g., deoxyribonucleic acid (DNA), ribonucleic acid (RNA), or amino acids. High-throughput sequence design refers to designing potentially very large numbers of sequence combinations from a set of input fragments. Each resulting output sequence is often referred to as a construct. Large collaborative teams of scientists and researchers can design and synthesize constructs or a derivative of the constructs in laboratory facilities in order to test and analyze the properties of the designed constructs High-throughput sequence design has applications in many different life sciences sectors, including antibody engineering, gene and cell therapy (e.g., CRISPR guide design), and strain engineering (e.g., cell line development, enzyme production).

### SUMMARY

This specification describes a bioinformatics platform implemented as computer programs on one or more computers in one or more locations that provides an enhanced user interface for automatic molecular sequence design. The enhanced user interfaces described in this specification can model both how fragments are prepared and how they are joined together in a single unified interface. In addition, the user interface designs described below are informed by real-world assembly methods, which greatly increases the likelihood of success of producing DNA sequences in a lab by cloning and helps model the modular design of more types of sequences by concatenation that can be further evaluated or synthesized in a lab.

Particular embodiments of the subject matter described in this specification can be implemented so as to realize one or more of the following advantages.

The user interfaces and associated techniques described in this specification provide an intuitive, easy-to-use, and biologically-informed way to generate molecular constructs from fragments, or a selected range of another sequence. The process is highly customizable for a particular design or for a particular cloning process, e.g., Golden Gate assembly, Gibson assembly, or homology techniques for DNA sequences, or concatenation, e.g., modularly joining or combining many DNA, RNA, or amino acid sequences.

The inputs and resulting outputs are easy to check and easy to visualize. In addition, the use of the interfaces and associated techniques for molecular construct generation creates an easily traceable record of how the construct information was generated and by whom. The techniques described below also provide the ability to limit possible fragment combinations and produce only select constructs and provide the ability to design, reuse, or design and reuse primers, e.g., DNA or RNA oligos, e.g., short sequences of DNA or RNA, that the user has previously designed when generating fragments for cloning, e.g., by polymerase chain reaction (PCR). These features result in more modular and reusable workflows, which is important for performing these activities at scale.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example bioinformatics platform.

FIG. 2 illustrates two example introduction modalities for the bioinformatics platform.

FIGS. 3A-3I illustrate example user interface presentations, e.g., within the context of cloning.

FIG. 4 illustrates an example user interface presentation, e.g., within the context of concatenation.

FIG. 5 demonstrates an example user interface presentation for saving constructs.

FIG. 6 is a flowchart of an example process for automatically generating construct combinations.

Like reference numbers and designations in the various drawings indicate like elements.

### DETAILED DESCRIPTION

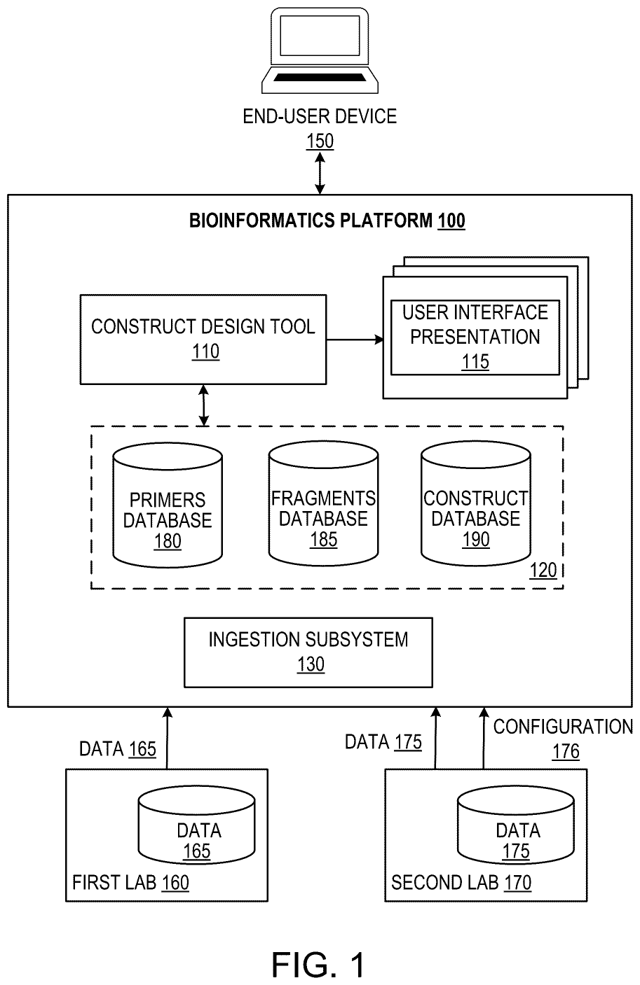

FIG. 1 is a block diagram of an example bioinformatics platform 100 that can generate a user interface presentation 115 for automated molecular construct design. The bioinformatics platform 100 is an example of a system implemented as computer programs on one or more computers in one or more locations in which the systems, components, and techniques described below can be implemented.

The bioinformatics platform 100 can be a distributed cloud-based computing system that includes: a construct design tool 110, a primers database 180, a fragments database 185, a construct database 190, and an ingestion system 130. The bioinformatics platform 100 can be used to assemble constructs in a combinatorial manner, e.g., leveraging either cloning or concatenation methods. In particular, cloning refers to the process of combining DNA sequences to produce new DNA sequences. As an example, Golden Gate assembly is a cloning technique that involves using Type IIS enzymes to reveal compatible “sticky ends” on two adjacent DNA fragments. As another example, Gibson assembly is a cloning technique that involves using primers to amplify DNA sequences, e.g., using PCR, such that two adjacent sequences have overlapping regions called homology regions. In this case, the homology regions are compatible when revealed by an exonuclease, an enzyme that chews back one strand of a two-stranded DNA sequence to expose a homology region for recombination.

The bioinformatics platform 100 can be used to assemble constructs for concatenation, e.g., the joining of one or more: (i) DNA sequences, DNA oligos, or both, (ii) RNA sequences, RNA oligos, or both, or (iii) amino acid sequences, respectively. In this case, the constructs can be combined, e.g., concatenated, for purposes other than cloning. In particular, the platform 100 can support the assembly of concatenation sequences by producing many output sequences from various combinations of input sequences.

In general, the bioinformatics platform 100 can use a data collection 120, which can include a primers database 180 and a fragments database 185, to automatically generate the sequences of molecular constructs using a construct design tool 110. The construct design tool 110 can generate user interface presentations 115, which can be provided to an end-user device 150. Upon receiving the sequences of molecular constructs, a user, e.g., a scientist, can perform one of the combinatorial assembly techniques, e.g., the cloning technique selected in the user interface, to synthesize the construct. In the case of cloning, a user can model and validate methods that they intend to perform in a lab and synthesize the construct sequences, e.g., directly in the lab or using a synthesis provider. In the case of concatenation, a user can design an RNA or amino acid construct as the end product that they aim to produce in the lab. The user can then determine which DNA sequence codes, e.g., using a back-translation method, for the RNA or amino acid sequence and then produce the DNA sequence, e.g., directly in the lab or using a synthesis provider.

Generally, the end-user device 150 can be an electronic device that is capable of requesting and receiving content over the network described above, e.g., the Internet. The end-user device 150 can include any client computing device such as a laptop/notebook computer, wireless data port, smart phone, personal data assistant (PDA), tablet computing device, one or more processors within these devices, or any other suitable processing device that can send and receive data over the network. For example, the end-user device 150 can include, e.g., a computer that includes an input device, such as a keypad, touch screen, or other device that can accept user information, and an output device that conveys information, including one or more of digital data, visual information, and the user interface presentation 115. The end-user 150 can include one or more client applications. A client application is any type of application that allows the end-user device 150 to request and view content on a respective client device. In some implementations, a client application can use parameters, metadata, and other information received, e.g., at launch, to access a particular set of data from the bioinformatics platform 100.

The bioinformatics platform 100 generally provides a centralized functionality for generating constructs. In particular, labs, e.g., groups or teams of scientists, e.g., the first lab 160 and the second lab 170, can upload data 165 and 175, respectively, through an ingestion subsystem 130 in order to store the data representing their molecular assets in the primers database 180 and fragments database 185. In some cases, the primers database 180 and fragments database 185 can be filtered using customized schemas, which can store additional properties of the sequences as custom metadata. As an example, the platform 100 can register the sequences and store properties as custom metadata fields, e.g., using a registry. The primers database 180 and fragments database 185 can also be filtered using standard, automatically captured data. As an example, the platform 100 can store who created or imported the sequence.

The ingestion subsystem 130 can be configured to perform data standardization transformations from the data received from different laboratories, e.g., in order to ensure their use across laboratories. Optionally, a lab, e.g., the first lab 160 or the second lab 170, can also specify configuration information 176 for customizing the behavior of the platform 100. In particular, this can include one or more of assembly set-up instructions, specifying the oligos or primers available to the lab at a current time, or indicating a certain configurable workflow process for use of the platform 100. In particular, the configurable workflow can include defining the fragments and the combinations to produce a specific set of constructs. As an example, customizing the behavior of the platform 100 can involve updating a schema that specifies the attributes displayed for each feature of the construct design tool 110. The ingestion subsystem 130 can thus be configured to receive the data 165 in any appropriate format, while the databases 180 and 185 can be configured to store the sequence data 165 in a particular database format e.g., the Hierarchical Editing Language for Macromolecules (HELM), as a string of nucleotides or amino acid residues, or any other appropriate format.

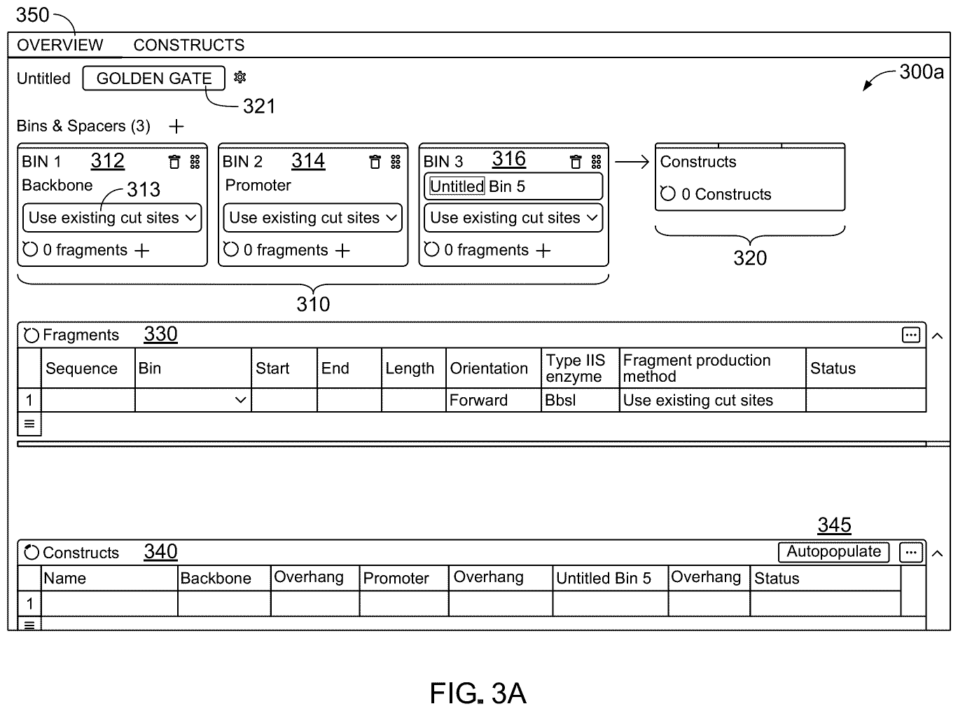

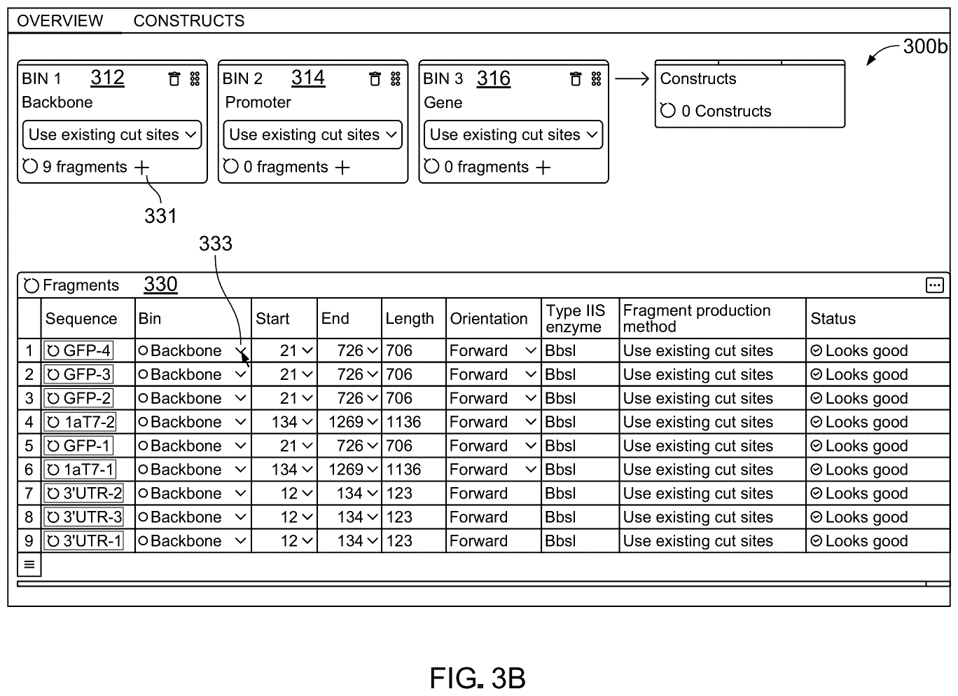

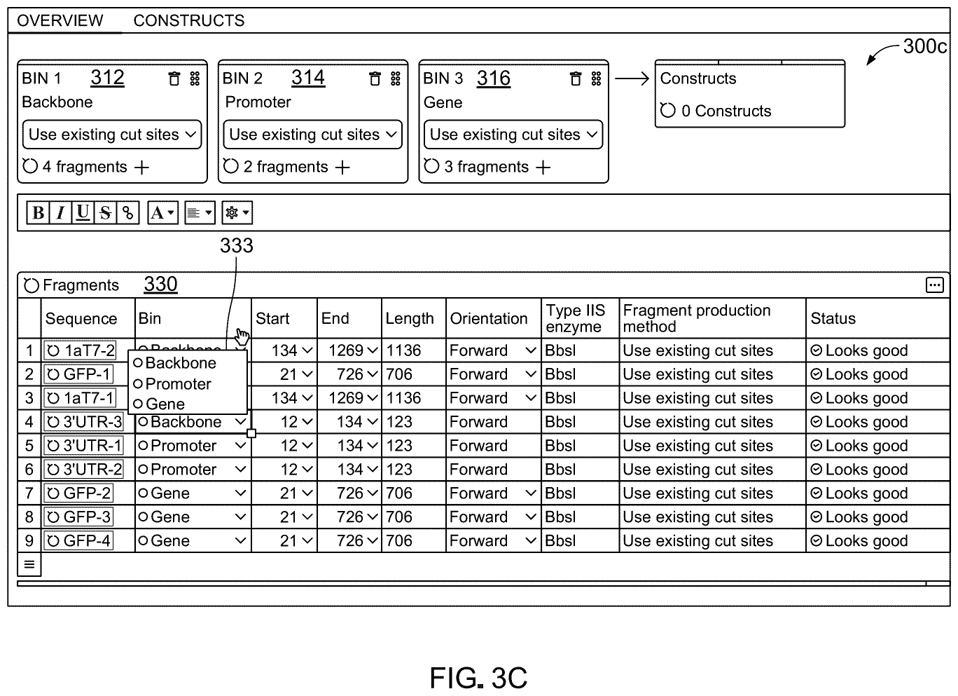

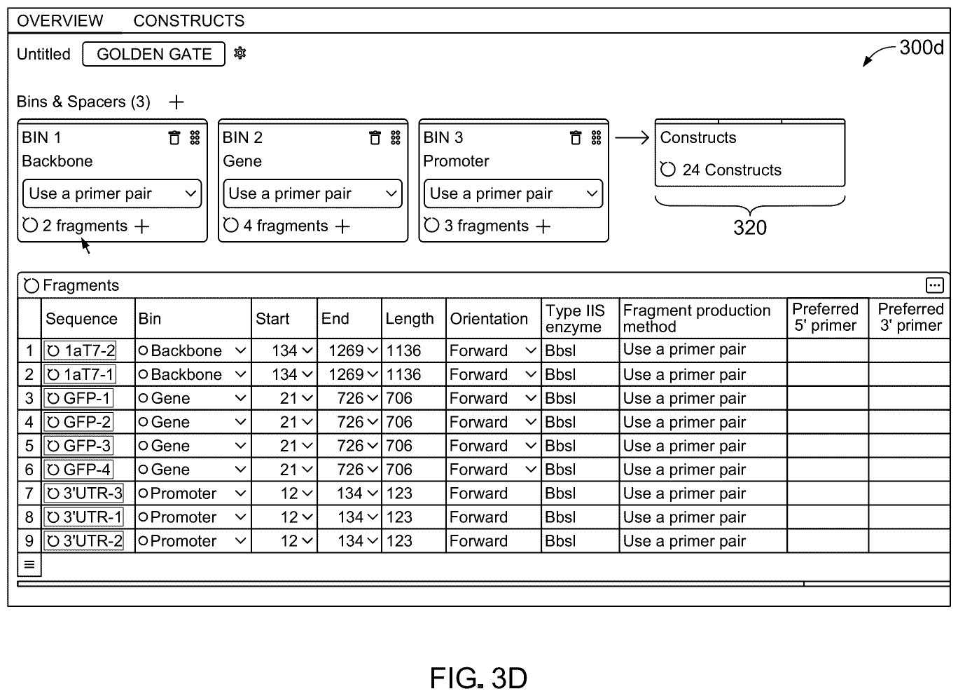

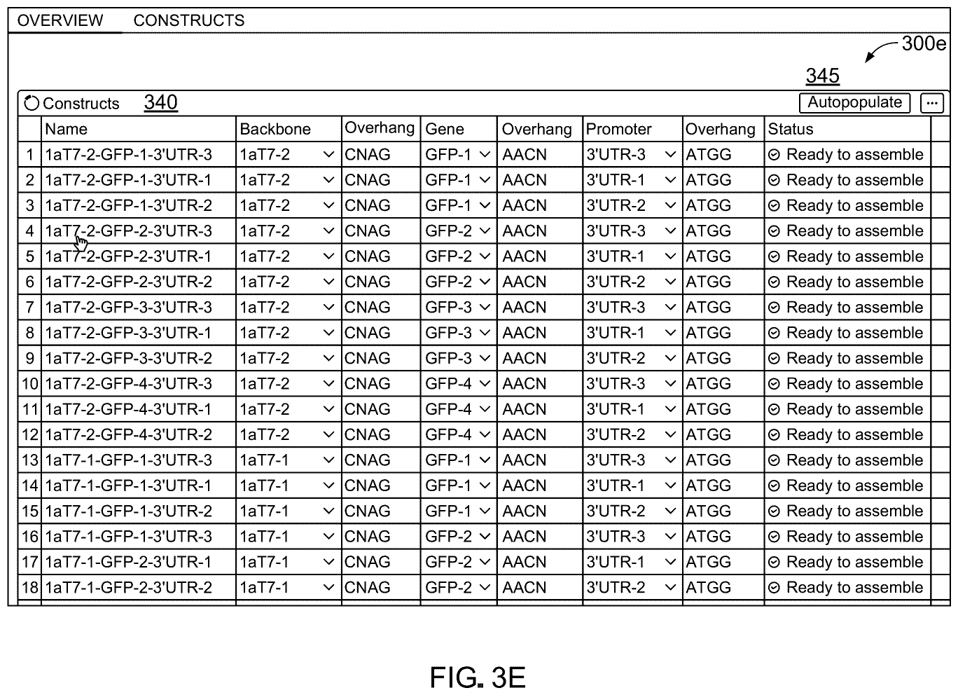

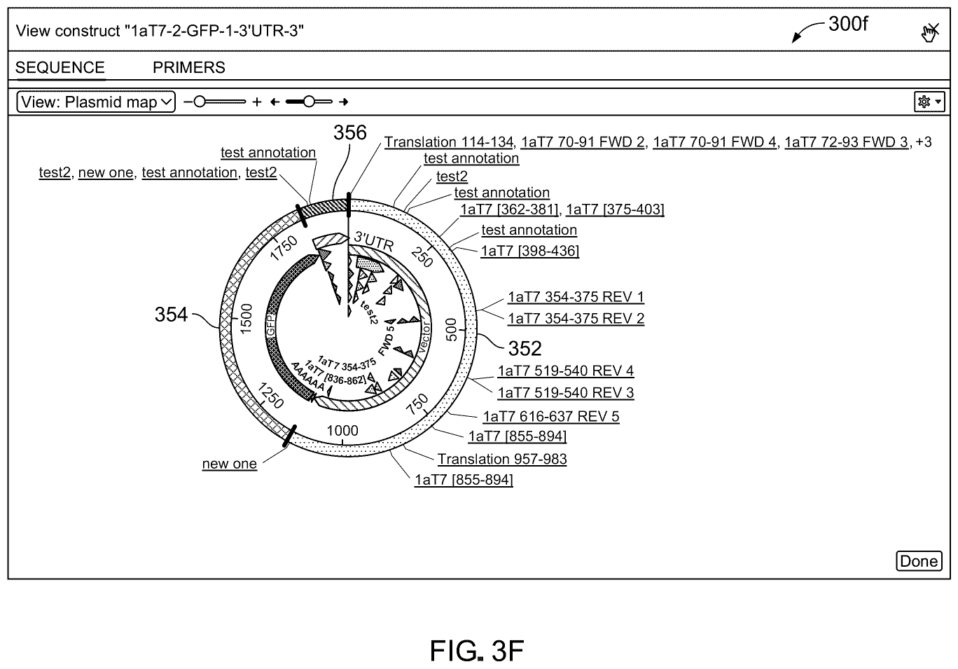

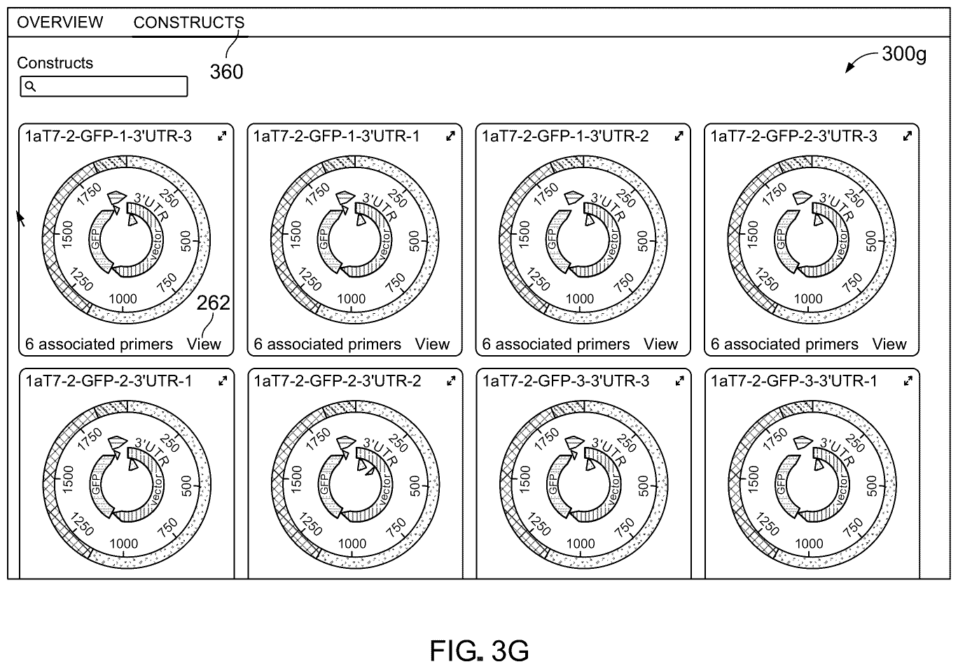

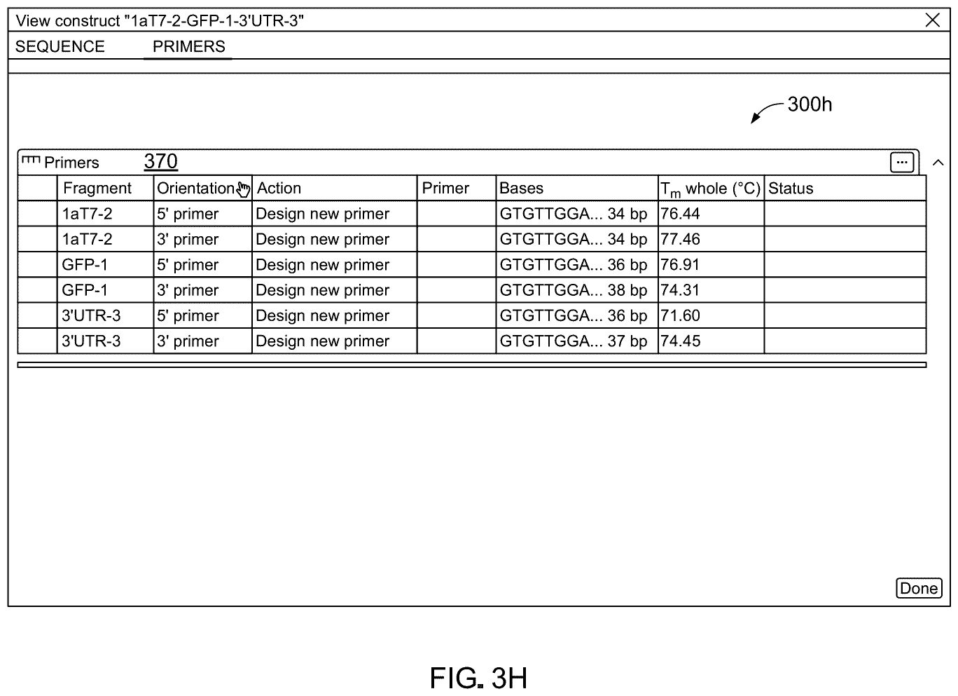

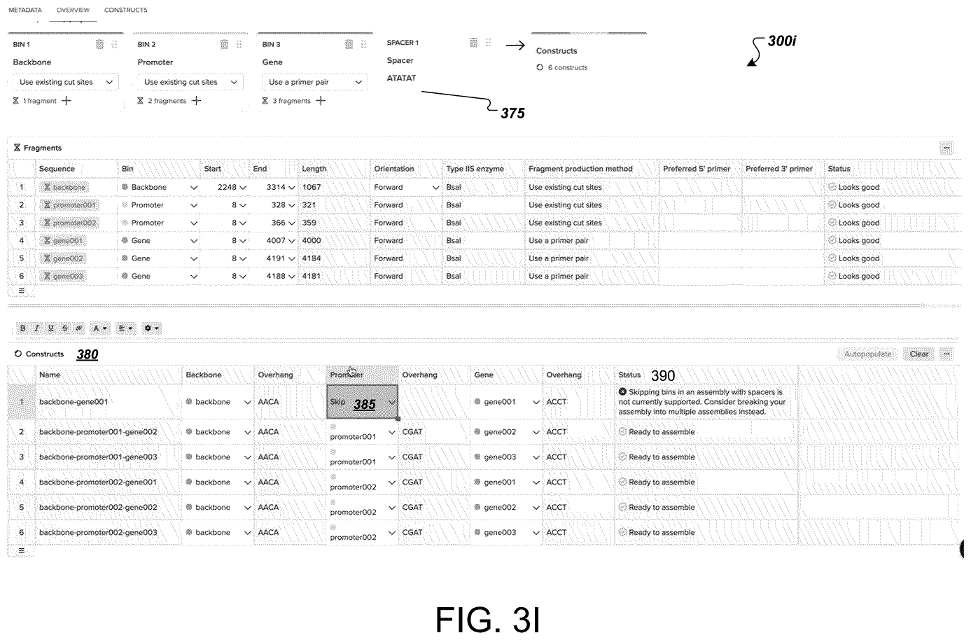

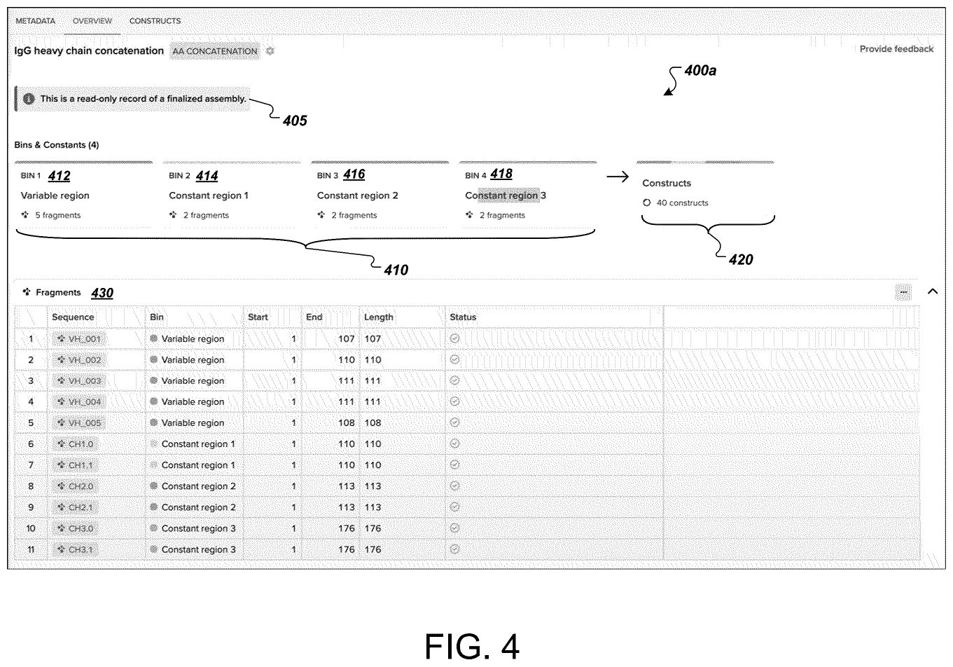

As described in more detail below with reference to FIGS. 3A-3H and FIG. 4, a user of the end-user device 150 can view the user interface presentation 115 and modify the user interface presentation 115 using one or more controls presented in the user interface presentation 115. For example, the user can interact with the controls to select one or more additional (or alternative) fragments or primers for generating constructs, e.g., using a fragments and constructs table, as described in further detail below. Furthermore, in some examples, the user can save their progress as they are performing an assembly, e.g., if the user leaves the combinatorial assembly tool before finalization, the data specified in the fragments table, constructs table, or both can be saved. In this case, the user can load the saved fragment or construct table in a new session to continue generating constructs from the loaded tables.

After generating the constructs, the user can add the generated constructs to a construct database 190, e.g., the user can finalize assembly and store the primers, fragments, and constructs. In particular, the construct database 190 can include DNA, RNA, or amino acid sequences, e.g., constructs previously created using the platform 100. In some cases, the construct database 190 can be organized by project, as will be described in further detail below. In some cases, the system can enable the filtering of types, e.g., DNA, DNA oligo, RNA, RNA oligo, or amino acid constructs, or searching for specific constructs, e.g., using an identifier. In some cases, the platform 100 can provide a file system to store the generated constructs in memory, e.g., in addition to storing the generated constructs in the database. In particular, the file system can be used to designate where constructs can be saved, e.g., at the creation of a cloning or concatenation project, and can locate and retrieve previously stored constructs, e.g., to serve as fragments in new assemblies or inform the generation of subsequent constructs.

As an example, the system can enable an easily traceable record of how the construct information was generated by providing a mechanism for saving metadata in the database 190. As another example, the system can enable the logging of a user identifier in the database 190 to easily associate the generator of the construct with the construct. In particular, the system can use filters based on the metadata to identify constructs, e.g., a user can access previously created constructs through a generic search tool in addition to finding them through assembly objects or records in the construct database 190. The construct database 190 can then be accessed using the construct design tool 110, e.g., a chosen construct can be identified from the construct database 190 and reloaded using the construct design tool 110. In some cases, the construct database 190 can receive data from elsewhere, e.g., additional connected platforms as part of a broader system.

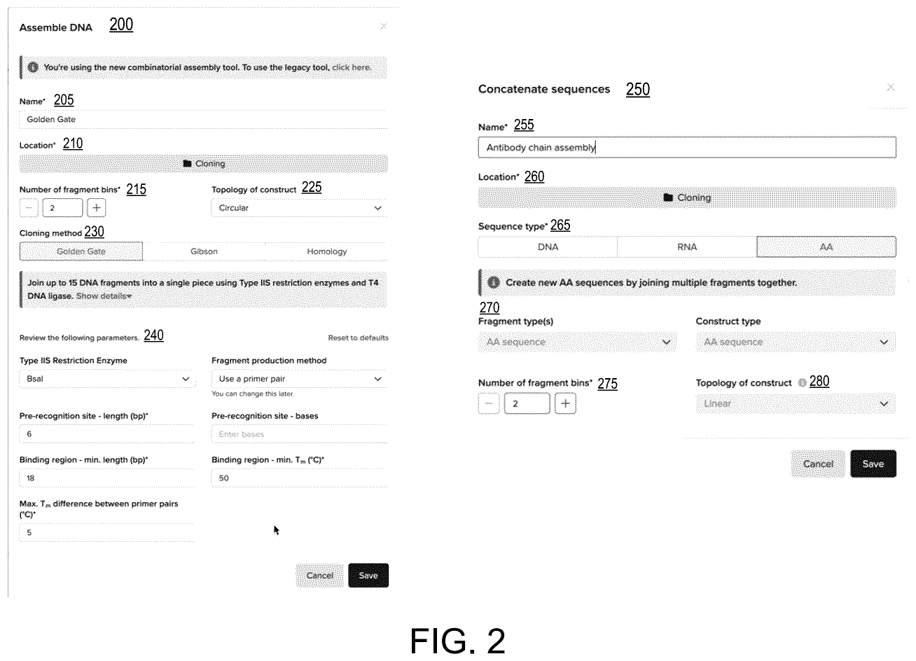

FIG. 2 illustrates an example introduction modality user interface presentation for a cloning method and a concatenation method. In particular, the user of the bioinformatics platform, e.g., the bioinformatics platform 100, can first elect whether they aim to generate constructs for cloning or concatenation by configuring the platform. In some cases, the introduction modality presentation is a window, e.g., a pop-up window. In particular, the platform can share the same assembly framework for both cloning and concatenation methods by applying different rules and configurations, e.g., as specified in the presentations 200 and 250.

In the case of cloning, the “Assemble DNA” presentation 200 can enable the user to input a name 205, e.g., a project name, for a cloning construct project. As an example, names 205 can be used to organize the generated and validated constructs, e.g., in the construct database 190 organized by projects. The presentation 200 also includes an input for saving location 210, e.g., the location where the constructs will be saved for the cloning construct project. In some cases, the platform can maintain a file system, e.g., in a computer-readable memory, such that the user can organize and maintain different projects under different file hierarchies. The user can also input the desired number of fragment bins 215, e.g., each bin corresponding to a different fragment, and the topology of the construct 225, e.g., circular for plasmid-based cloning methods.

The user can then select the cloning method 230, e.g., Golden Gate, Gibson, or Homology, and select related parameters 240. In the case of Golden Gate cloning, as depicted, the user can select the type of IIS restriction enzyme, the fragment production method, parameters related to binding, e.g., the pre-recognition site and binding region length, the pre-recognition site bases, and parameters related to the melting temperature Tₘ for PCR, e.g., the temperature at which half of the DNA nucleotides are in a single-strand state, e.g., the maximum difference between melting temperatures and the minimum melting temperature. An example user flow within the context of a cloning method will be described in more detail with respect to FIGS. 3A-3I.

In the case of concatenation, the “Concatenate sequences” presentation 250 can enable the user to input a name 255, e.g., a project name, for their concatenation construct project. Similarly, names 255 can be used to organize the generated and validated constructs, e.g., in a construct database 190 organized by projects. Likewise, the presentation 250 also includes an input for saving location 260, e.g., in a file system.

The user can select the sequence type 265 for concatenation, e.g., DNA, RNA, or amino acid (AA). In the particular example depicted, the user has selected amino acid as the sequence type 265. In this case, the related parameters 270, e.g., fragment type(s) and construct type are predetermined based on the user selection of amino acid. In the case that DNA or RNA are chosen for concatenation, the related parameters 270 can include an option for the fragments and constructs to be either DNA or RNA sequences or DNA or RNA oligos. The user can also input the desired number of fragment bins 275, e.g., each bin corresponding to a different fragment, and the topology of the construct 280, e.g., linear for concatenation. An example user flow within the context of a concatenation method will be described in more detail with respect to FIG. 4.

FIG. 3A illustrates an example user interface presentation 300a having an overview tab. The user interface presentation 300a is an example of a computer-implemented user interface presentation that can allow a user to perform automatic combinatorial sequence design, e.g., the arrangement of molecular material according to specified biological constraints, for the creation of constructs.

The user interface presentation 300a illustrates a sequence of bins 310 on the overview tab 350. In this example, the sequence 310 includes three bins 312, 314, and 316. Each bin represents a collection of fragments for generating constructs. As an example, the fragments can include a backbone, e.g., the alternating deoxyribose and phosphate groups of the DNA helix, a promoter, e.g., a segment of DNA that can be identified for the initiation of gene transcription using RNA polymerase, and gene sequences, e.g., the gene to be cloned from the DNA. In some cases, fragments can be reused within a bin or repeated across bins. In particular, if a user wants to use the same sequence in two different bins, the user can list it twice in the fragments table, e.g., with one row assigned to one bin and one row assigned to another.

To generate a construct, e.g., in a constructs table 340, the system can select a single fragment from each bin. To generate all possible constructs, the system can select every possible combination of selecting a single fragment from each bin, e.g., by selecting the auto-populate button 345 to automatically populate the constructs table 340. In some cases, a user can elect to skip a bin for a given construct. An example for skipping a bin will be covered in more detail in FIG. 3I.

The arrangement of the bins within the user interface presentation 300a represents an ordering of fragments in the generated constructs. Thus, in the sequence of the generated construct, a first fragment selected from the first bin 312 will be adjacent to a second fragment selected from the second bin 314, which will be adjacent to a third fragment selected from the third bin 316. In some cases, the sequence represents a circular strand of molecular material, e.g., a plasmid, in which case the third fragment is also implicitly adjacent to the first fragment.

The user interface presentation 300a allows a user to modify the ordering of the sequence of bins 310, for example, by dragging the bins around to change the order.

Each bin 312, 314, and 316, has a customizable bin name. In this case, the first two bins 312 and 314 are named “Backbone” and “Promoter” respectively. Selecting the name of a bin can allow a user to change the bin name. For example, the name of the third bin 316 has been selected, which allows a user to provide a new bin name.

In the particular example depicted, the user interface presentation also includes a construct counter 320, which provides an indication of the number of constructs that have been generated from the sequence of bins 310. In another example, the number of constructs or fragments is implied by the number of rows in the construct table 340, e.g., since each row has a number associated with it. In the example depicted in FIG. 3A, no constructs have yet been generated.

The presentation 300a also includes a fragments table 330 and a constructs table 340, which will be discussed in more detail below.

For cloning methods, in order to actually join molecular fragments in a lab, a particular joining technique must be used. The joining techniques are often dependent on the cloning technique that will be used in the laboratory to physically synthesize the construct. As an example, common joining techniques include using restriction enzymes to form sticky ends at specific cut sites, using primers to amplify DNA fragments with overlapping ends, and using Gibson assembly to anneal overlapping ends.

Each bin can thus include a selection element 313 for selecting the joining technique. In some implementations, the user interface presentation 300a also includes a selection element 321 for selecting the cloning technique itself, which can also populate a default joining technique that the user can change. For example, when selecting the Golden Gate cloning technique, the system can automatically populate the bins 312, 314, and 316, to use cut sites rather than primers. The user may then change the selection on one or many bins to use primers instead of cut sites. As another example, when selecting the Gibson assembly technique, the system can automatically populate the bins 312, 314, and 316, to use primers rather than cut sites or existing homology regions.

In this way, the user interface 300a models together two different aspects of cloning: the techniques for preparing the fragments and how the fragments are arranged, e.g., by the sequence of bins. For example, when a particular cloning methodology uses restriction enzymes, the user interface 300a can provide the ability to select or automatically populate certain restriction enzymes in order to model the corresponding process in the lab. For example, the molecular fragment can be prepared by cutting the fragment with the restriction enzyme. If the cut site is already present in the fragment, the user interface 300a can simply model just cutting the fragment. On the other hand, if the cut site is not present in the fragment, the user can introduce a cut site, e.g., which represents producing a fragment via PCR, where the primers contain a cut site and an overhang. Then, once the fragment has been produced, e.g., by PCR, a scientist can cut the molecular fragment with the restriction enzyme. After being cut, the sticky ends of adjacent fragments—if they are compatible—can be joined together in a way that is represented by the bin sequence.

In particular, in the case of cloning, compatibility can refer to the biochemically-allowable complementary pairing of an adenine (A) to thymine (T) in a DNA molecule, A to uracil (U) in an RNA molecule, or cytosine (C) to guanine (G) in a DNA or RNA molecule. In the case of Golden Gate assembly, Gibson assembly, and homology-based cloning methods (which take advantage of a biological process called homologous recombination), the system can take into account parameters that the user has set, like the length of the homology region, the binding region of a primer, or the melting temperature of a primer to determine whether the construct is valid. As an example, assessing the compatibility of Golden Gate fragments can involve making sure that overhangs exposed by a Type IIS enzyme are compatible and that primers, if used, have appropriately introduced enzyme cut sites with compatible overhangs. As another example, assessing the compatibility of Gibson fragments can involve making sure that the homology regions are compatible and that primers have appropriately introduced homology regions.

In the case of concatenation of DNA, RNA, or amino acid sequences, which does not involve primers, the system can string together sequences of compatible types.

FIG. 3B illustrates another example user interface presentation 300b. The example in FIG. 3B illustrates fragment selection.

Each bin in the sequence of bins 310 includes a fragment selector 331 that allows a user to import fragments for the design. The fragments can be imported from a variety of sources, e.g., a location identified from the file system, a centralized fragments database on the bioinformatics platform, an external database, or a text or a spreadsheet file, to name just a few examples. After importing the fragments, the system automatically populates the fragments table 330. In some cases, the system can only display fragments that can contribute to valid sequences, e.g., as defined by the fragments selected for other bins.

Each fragment in the fragments table 330 has a variety of attributes, including a sequence name, a bin assignment, a starting base pair, an ending base pair, a length. As another example, the attributes can include a DNA orientation, e.g., forward or reverse, type IIS enzyme, fragment production method, and status. These attributes can be customizable, e.g., as part of a schema a user submits to the system. For example, the attributes can be selected by a user when the user imports sequences into the system.

In particular, the status attribute indicates whether a row sequence has a warning or blocking error associated with it. For example, in the fragments table 330, the status indicates whether the fragment has been properly specified, e.g., all of the required columns are filled out with valid inputs. As an example, if the fragment is in a bin that uses a specific restriction enzyme, the system can ensure that the restriction enzyme cut site for that enzyme is in the fragment. If not, the system can indicate that the sequence will not bind using a status indicator. As another example, the status can be included in the constructs table, e.g., the constructs table 340, to indicate whether the construct has been properly specified and can be produced given the user's specifications. For example, if a fragment that is in the middle of the sequence only has one cut site, the system can use the status indicator to specify that the sequence only has one cut site and is not valid.

The user interface presentation 300b allows a user to easily and intuitively assign fragments to bins. In this example, the fragment selector 331 was selected for the Backbone bin 312. Thus, all the fragments in the fragments table were assigned to the Backbone bin. Each fragment in the fragments table 330 also includes a bin selector 333, which allows a user to reassign fragments to bins, e.g., bin 1 312, bin 2 314, or bin 3 314.

FIG. 3C illustrates an example user interface presentation 300c for reassigning fragments to bins. By using the bin selector 333, a user can change the bin assignments. Selecting the bin selector 333 causes the display of a new user interface presentation, e.g., the display of a pop-up selection window, that presents all of the bin names for bins in the sequence. In particular, the selection of the bin names can reassign a fragment to a bin.

As shown in FIG. 3C, after bin reassignment, the first bin 312 has been assigned four fragments, the second bin 314 has been assigned two fragments, and the third bin 316 has been assigned three fragments.

After the fragments have been assigned to bins and the joining techniques have been selected, the system can generate possible construct combination. In some cases, the system automatically generates every possible construct combination. In other cases, the user can elect to manually populate the fragments table, e.g., by selecting one or more fragments from each bin. As an example, the user can generate a particular construct with a specific combination of a backbone, promoter, and gene for experimental purposes.

FIG. 3D illustrates an example user interface presentation 300d for generating constructs. As shown in FIG. 3D, from bins containing 3, 4, and 3 fragments, the system can automatically generate 24 constructs, e.g., since the constructs are generated combinatorially. Because the constructs are generated combinatorially, each construct may also be referred to as a construct combination.

In addition to generating the constructs, the system can also automatically generate joining information that is needed to join the fragments in a lab, e.g., primers can be generated to create the desired sticky ends or restriction cut sites can be identified. This can include generating primer sequences for overhangs, e.g., sticky ends, and homology regions, e.g., for use in homologous recombination in which the ends of DNA fragments are modified to expose single stranded DNA that is similar or identical across molecules. Where the exposed ends are compatible, e.g., complementary, with the exposed ends of other fragments, the DNA can recombine or rejoin. Other cloning techniques can also take advantage of this process, which requires identifying and using regions of complementary molecular material at the ends of the adjacent fragments. Generating the joining information can also include finding existing compatible ends and validating that all adjacent fragments can be joined together according to principles of the cloning methodology being modeled.

Notably, the system can automatically generate the joining information according to which fragments are being joined. For example, the primer that needs to be used to join two sequences is often highly dependent on the sequences themselves. The system can thus use previously generated mappings between fragment pairs and primers and then use those mappings at construct generation time in order to automatically generate the joining information.

In this way, the system can model both the production of the fragments themselves as well as how the fragments can be combined in a biologically-informed way. In particular, the system can generate new information, e.g., primers for each fragment pair, and use attributes of each fragment sequence, e.g., existing cut sites in the sequence, in order to validate the construct combinations. In other words, instead of merely generating combinations of inputs, the system can use aspects of the particular cloning methodology in order to ensure that the corresponding molecular fragments are likely to be producible in a lab.

FIG. 3E illustrates an example user interface presentation 300e having a populated constructs table 340. The constructs table 340 presents each construct, e.g., each construct generated according to the fragments chosen in FIG. 3D, with a construct with a construct name and a number of other attributes. The constructs table 340 can be presented in the user interface presentation 300e below the fragments table 330 and bin sequence or can be presented in a separate interface.

The constructs table 340 also includes a status attribute. The status attribute represents the results of one or more validation procedures performed on the generated constructs. For example, the system can perform error checking on the automatically generated constructs to ensure that they do not violate one or more known rules about how constructs are created. In particular, the system can computationally model one or more of restriction enzyme digestion, sequencing, PCR amplification, or gel electrophoresis to validate the resultant construct sequence.

FIG. 3F illustrates an example user interface presentation 300f for visualizing a construct. For example, a user can select or hover over a construct in the constructs table 340, and in response, the system can generate a visualization of the selected construct. In this example, the visualization is a plasmid map, although any other appropriate visualization can also be used depending on the type of construct. In particular, in the case that a linear sequence is being produced, the visualization can display a linear sequence map.

The user interface presentation 300f visually distinguishes different fragments of the construct in order to represent from which bin the fragment was selected. For example, the system can use color coding by matching the color of the bin with the color of the fragment in the visualization. In this example, the first fragment 352 was selected from the backbone bin 312, the second fragment 354 was selected from the gene bin 314, and the third fragment 356 was selected from the promoter bin 316.

FIG. 3G illustrates an example user interface presentation 300g having a constructs tab. In particular, a user can select the constructs tab 360, and, in response, the system can generate a scrollable visualization of all construct combinations. The visualizations can include information to relate each fragment of each construct combination back to the bin from which it originated, e.g., color coding, legends, or pattern coding. The visualizations can also display joining information, e.g., the primers or restriction engine cut sites that are being used.

For example, each visualization can have a selector 362 that causes the system to display more information about the joining technique that is being used. The user can select the selector 362 to view the sequence in more detail, e.g., one or more of the base sequence, complementary sequence, and an indication of modification of bases and sugars.

FIG. 3H illustrates an example user interface presentation 300h that displays primer information. The user interface presentation 300h is an example of a user interface that can be presented in response to a user selecting the join technique selector 362 shown in FIG. 3G.

In this example, the user interface presentation includes a primers table 370 that lists each of the primers for the construct combination along with a set of primer attributes. In the particular example depicted, the table 370 includes information about the orientation, e.g., whether the primer is oriented in the 5′ or 3′ direction, the DNA or RNA bases included in the primer, and the melting temperature Tₘ for PCR, e.g., the temperature at which half of the DNA strands are in a single-strand state.

This can be very useful information because not all facilities have every primer on hand at all times. As an example, the user interface can allow a user to filter the primers table 370 based on available primers, e.g., which can be input into the system as part of customizing the platform as discussed in FIG. 1. As another example, the user interface can communicate which of the primers in primers table 370 have been designed by the system and which were added by the user.

In particular, a “preferred primer” column can indicate a primer created by the user, which can be saved as an oligo in the construct database 190 and loadable and selectable as a clickable entity within the table 370. If preferred primers are not found with respect to the fragments chosen, the platform 100 can generate suggested primers, which can be saved as constructs once assembly is finalized. For example, the system can support searching and filtering features in a centralized primers collection, which can allow the user to both search for primers that are available for the construct combinations, as well as to list only the construct combinations that can be synthesized with the primers that are already on hand. Therefore, molecular biology teams can begin work immediately while other primers for building the other constructs are acquired.

FIG. 3I illustrates an example user interface presentation 300i that includes an option to add a spacer sequence in a construct. In particular, FIG. 3I illustrates an example spacer bin 375 that enables a user to include a spacer sequence, e.g., the sequence ATATAT. As an example, a spacer sequence can be a region of non-coding DNA, e.g., a number of bases between genes.

In particular, the system can include the spacer with a primer pair, e.g., the pair of primers that determine which sequence of DNA gets amplified in PCR. As an example, the system can require that a primer pair be included in the construct in order to use the spacer. In the case that the user elects to skip the promoter 385, e.g., by toggling the promoter off in the constructs table 380. While this invalidates the construct in this case, e.g., as displayed by the indicator in the status column 390, in some cases the user can elect to skip the promoter 385 to assess the overhang between the gene and the backbone. In particular, the user can assess whether the sticky ends of the backbone and the gene are compatible without the promoter.

FIG. 4 illustrates an example user interface presentation 400a having an overview tab. The user interface presentation 400a is an example of a computer-implemented user interface presentation that can allow a user to perform automatic combinatorial sequence design for concatenation.

The user interface presentation 400a illustrates a sequence of bins 410 on the overview tab 450. In this case, the user has loaded a record 405 of constructs 420, e.g., from the construct database. In this example, the sequence 410 includes four bins, e.g., bin 412, 414, 416, and 418. Similarly to the cloning example described in FIG. 3A, each bin represents a collection of fragments for generating constructs. Since this is a record 405, the user previously assigned fragments, e.g., from a fragments table 430, to each bin 410, e.g., by adding to bins as previously discussed.

In the particular example depicted, the concatenation is for the formation of an antibody, e.g., an IgG antibody. In particular, the bins 414, 416, and 418 are constants, e.g., constant regions of DNA or RNA base, or amino acid residue sequences, that the user specified at the time of the generation of the construct. Constant bins can be used to add the constant sequences of bases or residues to every single construct, e.g., all 40 constructs in the particular example depicted. As an example, a constant can be a specified DNA oligo of “AAUG” or an amino acid residue sequence of “WST” for tryptophan, serine, and threonine.

For concatenation methods, the user can modularly design a set of sequences for concatenation. Depending on the sequence type, the user can convert to another sequence. As an example, in the case that the user generated an RNA, RNA oligo, or amino acid sequence using concatenation, the user can backtranslate to DNA and synthesize the sequence using DNA in order to synthesize the RNA sequence through transcription or the amino acid sequence through translation. In the case of DNA, the user can synthesize the sequence using the concatenated sequence, e.g., without backtranslating.

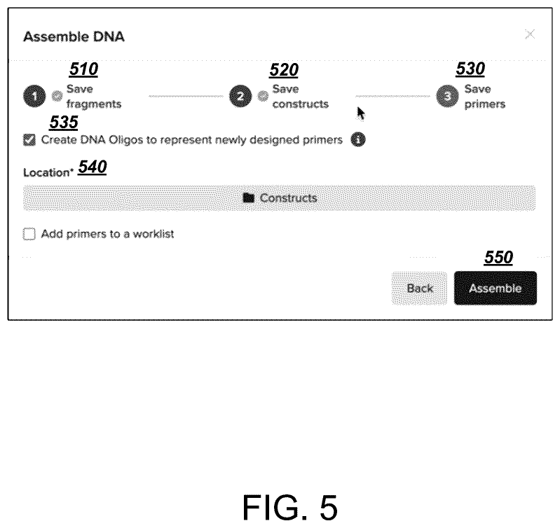

FIG. 5 demonstrates an example user interface presentation for saving constructs, e.g., after construct generation. In particular, the user of the bioinformatics platform, e.g., the bioinformatics platform 100, can save the generated sequences for cloning or concatenation using the presentation 500. In some cases, the presentation 500 is a window, e.g., a pop-up window.

The particular example depicted is for saving DNA from a cloning project. In particular the presentation 500 can lead the user through a user flow for saving the generated constructs and associated data. In this case, the user flow can include an option to save fragments 510, save constructs 520, and save primers 530. As an example, within the context of an example project, the user can save the fragments table, e.g., the fragments table 330, the constructs table, e.g., the constructs table 340, and the primers generated, e.g., either user-created or system-generated, in a location 540. In the particular example depicted, the location 540 can be selectable from a file system such that the data can be saved to memory. In some cases, the fragments, constructs, and primer data can additionally be stored in a database, e.g., the constructs database 190.

In particular, in the case of saving primers, the user can additionally elect to create DNA oligos representing the newly designed primers 535. As an example, these primers can be saved as DNA oligos. In this case, the user can access and reuse the DNA oligos, e.g., within the context of another project, by using that DNA oligo as a “preferred primer” object.

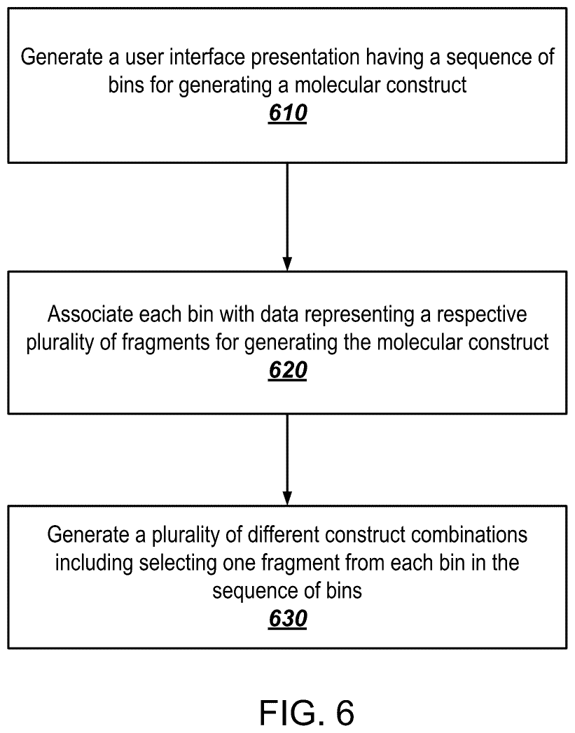

FIG. 6 is a flowchart of an example process for automatically generating construct combinations. The process can be performed by a system of one or more computers in one more locations and programmed in accordance with this specification. The process will be described as being performed by a system of one or more computers.

The system generates a user interface presentation having a sequence of bins for generating a molecular construct (step 610). As described above, a user can rename and reorder a sequence of bins for generating the construct.

The system can associate each bin with data representing a respective plurality of fragments for generating the molecular construct (step 620). For example, a user can import a collection of fragments for each bin. A user can also select particular cloning techniques and one or more joining techniques.

The system can generate a plurality of different construct combinations including selecting one fragment from each bin in the sequence of bins (630). The system can automatically generate every possible combination of constructs that can be generated by selecting one fragment from each bin. As described above, the system can also perform automatic validation of the construct combinations so that fragment pairs that are known to be incompatible, e.g., not complementary based on sequence, will not be produced in the final output. In some implementations, the system displays the resulting constructs in a constructs table, visualizations of the constructs, or both.

As part of this process, the system can automatically generate joining information for joining pairs of fragments. For example, the system can store a mapping between sets of one or more primers and pairs of adjacent fragments requiring each set of one or more primers. Then, when generating the construct combinations, the system can use the mapping to determine which primers are needed for which adjacent fragments.

The system can also support various searching features that enable teams of researchers to rapidly determine which constructs can be synthesized with materials on hand, e.g., available primers, and which materials are needed. For example, the system can automatically search a database representing a primers collection to determine which primers are available to be used and which primers need to be acquired to synthesize the construct. The system can generate a report indicating which constructs can be synthesized with materials on hand, and which primers or fragments need to be acquired in order for a construct to be synthesized. As the acquisition process can take time, this allows the teams to immediately commence synthesis of construct combinations for which the primers are already available.

The system can also support additional functionality for customizing the generation and registration of construct combinations. For example, the system can provide an interface for users to define a naming scheme or convention for constructs combinations so that they can be identified and referred to in a principled way. In addition, the system can provide an interface by which users can define rules that tell the system which combinations of fragments to generate or not generate, e.g., which pairs of sequences to combine or not combine. Lastly, the system can provide users with a tool for mapping generated constructs to system schemas so that the generated constructs can be easily or automatically added to a customer's construct database or registry after creation.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method comprising:
generating a user interface presentation for automatically generating different combinations of molecular sequences, wherein the user interface presentation presents a sequence of bins for generating a molecular construct;
associating each bin with data representing a respective plurality of fragments for generating the molecular construct; and
generating a plurality of different construct combinations representing different variations of the construct, including selecting, for each different construct combination, one fragment from each bin in the sequence of bins.

2. The method of claim 1, wherein the user interface presentation models both how the fragments are prepared and how they are joined together.

3. The method of claim 1, further comprising presenting, within the user interface, a representation of each of the plurality of different construct combinations.

4. The method of claim 3, wherein presenting the representation of each of the plurality of different construct combinations comprises populating the user interface presentation with a constructs table that presents data representing each different construct combination.

5. The method of claim 4, further comprising generating, within the user interface presentation, a fragments table that lists all the fragments in all of the multiple bins.

6. The method of claim 5, further comprising receiving user input in the presentation of the fragments table that changes the bin association of a fragment in the fragments table.

7. The method of claim 6, further comprising:
storing a mapping between pairs of adjacent fragments and data representing how each pair of adjacent fragments are joined.

8. The method of claim 6, further comprising generating, for each construct combination, data representing how each pair of adjacent fragments in the construct combination are joined.

9. The method of claim 7, wherein the data representing how each pair of adjacent fragments are joined comprises data representing what restriction cut sites to use and what overhangs or sticky ends are produced, which primers to use, or what homology regions are used or should be generated.

10. The method of claim 9, wherein the data representing which primers to use represents which primers to use to amplify a particular fragment or introduce cut sites, homology regions, or other properties of the fragment.

11. The method of claim 10, further comprising:
receiving user input selecting a particular construct combination; and
generating a visual representation of the particular construct combination.

12. The method of claim 11, wherein the visual representation has multiple segments, each segment corresponding to a different respective bin of the sequence of bins.

13. The method of claim 12, wherein the sequence of bins has a respective associated color, and wherein the multiple segments of the visual representation of the particular construct combination have colors matching colors of the sequence of bins.

14. The method of claim 11, wherein the visual representation of the particular construct combination comprises a visual representation of primers required to join adjacent fragments of the construct combination.

15. The method of claim 14, further comprising:
receiving a user input selecting a particular primer used for a particular construct combination; and
searching a repository representing available primers; and
generating a report indicating whether the primer is represented in the repository.

16. The method of claim 15, further comprising receiving user input specifying a naming convention for the construct combinations, wherein generating the plurality of different construct combinations comprises generating corresponding names according to the naming convention.

17. The method of claim 16, further comprising receiving user input specifying one or more rules for construct combinations that should be created or that should not be created, wherein generating the plurality of different construct combinations comprises generating the construct combinations according to the one or more user-specified rules.

18. The method of claim 17, further comprising obtaining one or more system schemas for generated constructs and adding the generated construct combinations to a construct database or registry according to the one or more system schemas.

19. A system comprising one or more computers and one or more storage devices storing instructions that are operable, when executed by the one or more computers, to cause the one or more computers to perform operations comprising:
generating a user interface presentation for automatically generating different combinations of molecular sequences, wherein the user interface presentation presents a sequence of bins for generating a molecular construct;
associating each bin with data representing a respective plurality of fragments for generating the molecular construct; and
generating a plurality of different construct combinations representing different variations of the construct, including selecting, for each different construct combination, one fragment from each bin in the sequence of bins.

20. A computer storage medium encoded with a computer program, the program comprising instructions that are operable, when executed by data processing apparatus, to cause the data processing apparatus to perform operations comprising:
generating a user interface presentation for automatically generating different combinations of molecular sequences, wherein the user interface presentation presents a sequence of bins for generating a molecular construct;
associating each bin with data representing a respective plurality of fragments for generating the molecular construct; and
generating a plurality of different construct combinations representing different variations of the construct, including selecting, for each different construct combination, one fragment from each bin in the sequence of bins.
